(19) United States
(12) Patent Application Publication
(10) Pub. No.: US 2014/0295008 A1
(43) Pub. Date: Oct. 2, 2014

(54) System and Method for Time-Release of Ellagitannins

(71) Applicant: NutraBerry LLC, Kenmore, WA (US)

(72) Inventors: David M. Wishnick, Seattle, WA (US); Robert G. Allan, Kenmore, WA (US)

(73) Assignee: NutraBerry LLC, Kenmore, WA (US)

(21) Appl. No.: 14/224,480

(22) Filed: Mar. 25, 2014

Related U.S. Application Data
(60) Provisional application No. 61/853,074, filed on Mar. 28, 2013.

(51) Int. Cl. A61K 36/73; A61Q 19/10; A61K 8/97

(52) U.S. Cl. CPC A61K 36/73; A61K 8/97; A61Q 19/10
USPC 424/765; 424/776

(57) ABSTRACT

Disclosed herein are embodiments of compositions that include a plurality of seed particles of various sizes, which may be desirable for providing the time-release of compounds present in the seed particles. By having compositions with seed particles having a range of sizes, various desirable time-release profiles may be generated by varying the range of sizes of the seed particles and varying the distribution of various sizes.

### CROSS-REFERENCE TO RELATED APPLICATIONS

This application claims the benefit of U.S. Provisional Application No. 61/853,074, filed Mar. 28, 2013, which application is hereby incorporated herein by reference in its entirety for all purposes.

### BACKGROUND

Among the numerous plants that have medicinal properties, cane-berries, including raspberries, are particularly noteworthy because of their content of ellagitannins. Many scientific articles on the biological activity of the components of the cane-berries have been published. Some of these studies have shown the physiological health benefits of ingesting whole cane-berries with regard to the inhibition of colon cancer in humans. The effective dosage suggested by some studies is about 300 grams of raspberries per day for a human weighing about 180 pounds. Each year, in the United States alone, about 50,000 people are diagnosed with this life-threatening cancer. The mechanism by which ingested cane-berries inhibit some types of cancer is likely correlated with the substantial presence of ellagitannins in these berries.

One of the difficulties of employing ellagitannins and other antioxidants medicinally in the form of whole berries is achieving an effective dosage. Whole berries contain about 82% moisture, so a large portion of the fruit is simply water. This means that a patient must consume a considerable amount (roughly 16 cubic inches) of berries each and every day for medicinal efficacy. In human feeding protocols this can become a tiresome task that many patients are unable to sustain. The need for such a large ingestion of berries is because only about 10% the total ellagitannins in the total fruit is located within the flesh of the berry and biologically available. The other 90% is located within the seeds which normally pass through the body intact and therefore do not give up the contained ellagitannins for absorption by the body. Although ellagitannins are present in sufficient quantities in seeds, they are not nutritionally available because of the form in which the seeds enter the body, and therefore the ellagitannin-rich seeds pass through the body without releasing ellagitannins in a medicinally sufficient amount.

Furthermore, it is well recognized in the literature that the effectiveness of any drug or medicine is typically enhanced when it is delivered repeatedly over a period of time. This is because when a compound is administered in one single dose, the level of that compound in the body increases rapidly and linearly through the minimum effective level to a peak value. The medicine is then consumed within the body and the level thereafter declines rapidly and exponentially by first order kinetics. When the declining level reaches the minimum effective concentration again, the compound ceases to have efficacy as a medicine.

The period of effectiveness of the medicine is the elapsed time between these two ascending and descending points on the minimum effective line and is usually rather short in duration. Conventionally, the only way to overcome such undesirable decay considerations is taking a second dose of the medicine within a short time period to again raise the level of the medicine within the body to above the minimum effective concentration. Controlled release delivery systems for medicines provide one solution for this problem. When the medicine is in such a form, the continuous, incremental delivery of a new small quantity of the medicine from a reservoir replaces amounts of the medicine which have been lost by consumption or decomposition within the body and therefore maintains the critical effective level of the medicine within the body.

These controlled release delivery systems take a variety of forms. Some methods include a system where the active medicine is physically encased within a plastic or other compound from which the medicine slowly escapes over a period of time by diffusion. Another delivery method includes a system where the active medicine is chemically reacted with a plastic or other compound to become a new substance that becomes temporarily biologically unavailable. However, over time and after ingestion by a subject, the temporary chemical linkage can be cleaved by the chemistry of the body to release the medicine in its active form. However, such delivery systems are not suitable for the administration of ellagitannins from cane-berries. Such processing results in an “un-natural” product which is undesirable to may consumers.

Without a controlled release delivery system, a patient would have to eat berries continuously, throughout the day and night, to achieve maximum results with the minimum dosage. Clearly, such a regimen poses a tremendous patient compliance challenge. Accordingly, there is a need for a controlled release delivery system for the administration of the active principles present in cane-berry seeds and for raspberries in particular.

### BRIEF DESCRIPTION OF THE DRAWINGS

FIG. 1a is an exemplary drawing illustrating an embodiment of seed particles of different sizes.

FIG. 1b is an exemplary drawing illustrating an embodiment of seed particles consisting of a single unit and comprising a plurality of sub-units.

FIGS. 1c-1e depict example seed particle size histograms in accordance with some embodiments.

FIGS. 2a-2d depict time-release profiles in accordance with some embodiments.

FIG. 3 is a block diagram of a method for generating a time release composition from plant material comprising seeds, in accordance with an embodiment.

FIGS. 4a-4c are block diagrams of methods for generating time release compositions from processed seeds, in accordance with some embodiments.

FIG. 5 is a block diagram of a method for time release of ellagitannins in a subject.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

Since currently-available time-release and controlled delivery systems are not suitable for the time-release and controlled delivery from cane-berries, a natural composition of cane-berry seeds that provides for the time-release and controlled delivery of ellagitannins can prove desirable and provide a basis for a wide range of therapeutic and nutritional products. This result can be achieved, according to various embodiments of systems and methods for time-release of ellagitannins that are shown and described herein.

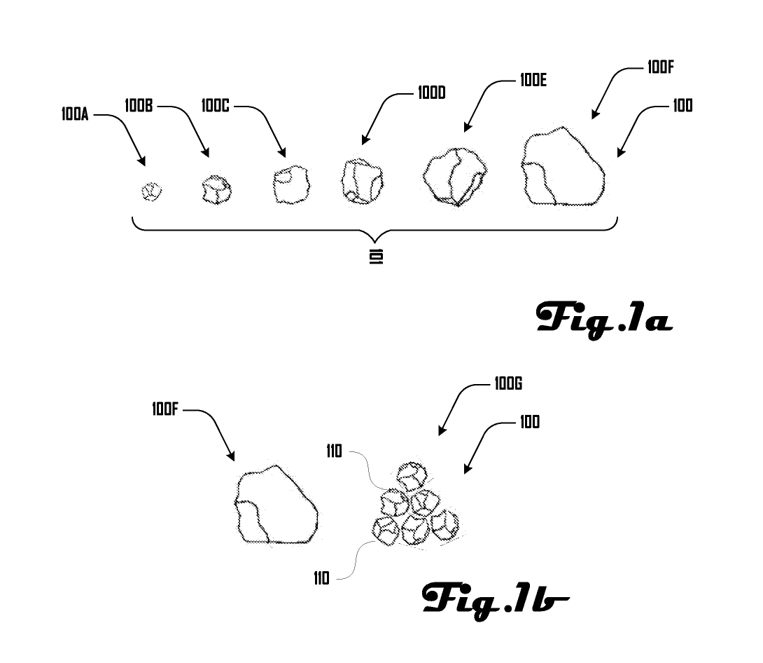

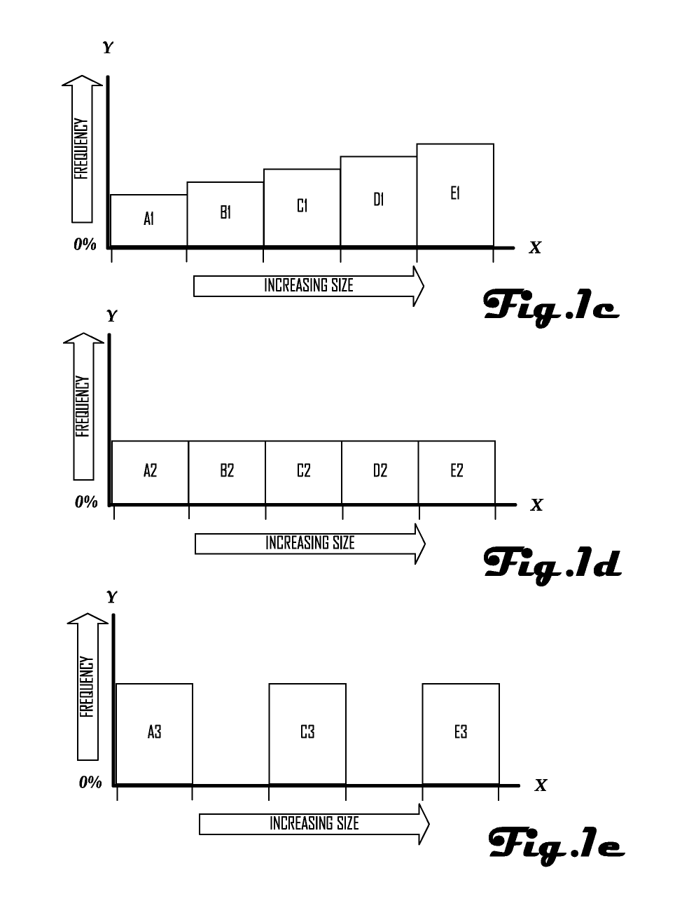

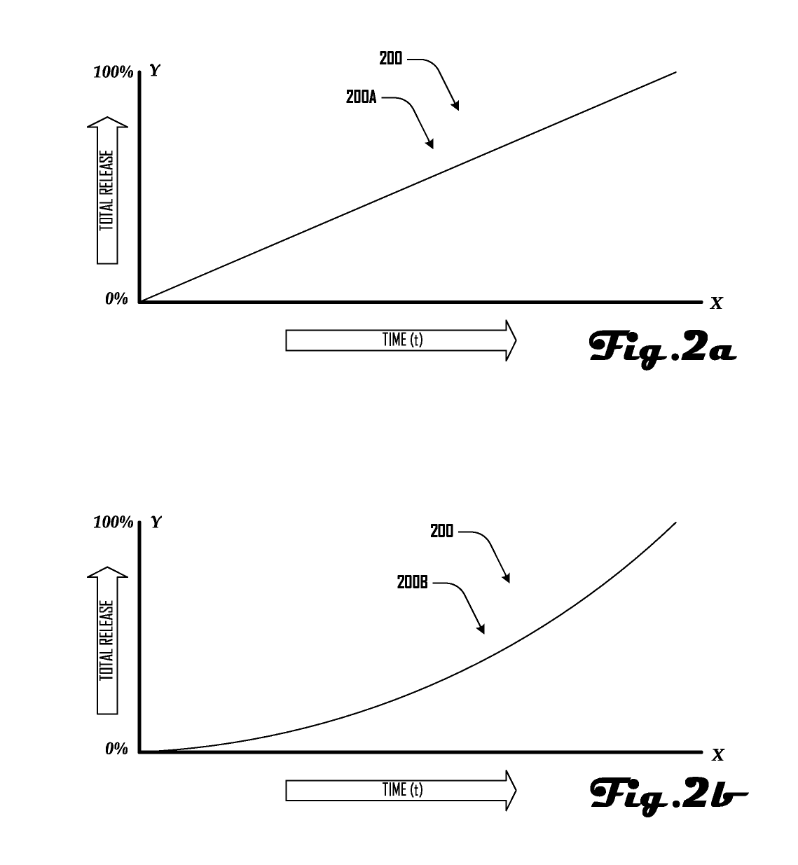

Turning to FIG. 1a, an example composition 101 having seed particles 100 of different sizes is depicted. For example, FIG. 1 depicts six seed particles 100A-100F of increasing size. The seed particles may be a single unit as depicted by seed particles 100A-100F. In some embodiments, seed particle 100 may comprise a plurality of sub-units 110 as depicted by seed particle 100G of FIG. 1b. While many embodiments discussed herein include seed particles 100, in further embodiments, any suitable plant material comprising desirable compounds may be used in a composition in accordance with the present invention.

In various embodiments, compositions may have a plurality of seed particles 100 of various sizes, which may be desirable for providing the time-release of compounds present in the seed particles 100. By having compositions with seed particles 100 having a range of sizes, various desirable time-release profiles may be generated by varying the range of sizes of the seed particles 100 and varying the distribution of various sizes. For example, FIGS. 1c-1e depict example seed particle size histograms in accordance with some embodiments. As depicted in FIG. 1c, for some compositions, distribution of sizes may include a greater numbers larger particles compared to smaller particles, with the particle frequency increasing with the size of particles. However, for some compositions, the size distribution may be substantially constant among all sizes as depicted in FIG. 1d. In further compositions, certain ranges may be completely excluded as depicted in FIG. 1e. Accordingly, FIGS. 1c-1e only depict examples of size distributions in accordance with some embodiments, and are not intended to limit the wide variety of size distributions that are within the spirit and scope of the present invention.

In some embodiments, a range of sizes can include the size of whole seeds to 5 μm. In accordance with some embodiments, size ranges of seed particles 100 may be defined by standard Mesh dimensions. In other words, a composition may be defined by a percent of total composition mass that will pass through one or more Mesh dimensions. As used herein the term “Mesh” in this disclosure refers to US Standard Mesh sizes as defined on the date of this filing. However, in some embodiments, other mesh standards may be used, including Tyler Standard mesh, Tensile Bolting Cloth Standard mesh, Mill Grade Standard mesh, and Market Grade Standard mesh, or the like.

For example, in one composition embodiment, 95-100% of the composition will pass through 40 Mesh; 60-80% will pass through 60 Mesh; 40-60% will pass through 70 Mesh; 1-10% will pass through 100 Mesh; and 0.1 to 1.0% will pass through 200 Mesh.

In various embodiments, there may be portions of a composition that are configured to pass and not pass through one or more Mesh of different sizes. In the example composition above, there are six Mesh sizes of 40, 60, 70, 100 and 200 Mesh. In further embodiments, the one or more Mesh size used to define a composition can include Mesh 7/16, ¼, 3.5, 4, 5, 6, 7, 8, 9, 10, 12, 14, 16,18, 20, 25, 30, 35, 40, 45, 50, 60, 70, 80, 100, 120, 140, 170, 200, 230, 270, 325, 400, 500, 632, or the like.

In some embodiments, a set that defines a composition consisting of three Mesh sizes may include: {40, 45, 50} {40, 45, 60} {40, 45, 70} {40, 45, 80} {40, 45, 100} {40, 45, 120} {40, 50, 60} {40, 50, 70} {40, 50, 80} {40, 50, 100} {40, 50, 120} {40, 60, 70} {40, 60, 80} {40, 60, 100} {40, 60, 120} {40, 70, 80} {40, 70, 100} {40, 70, 120} {40, 80, 100} {40, 80, 120} {40, 100, 120} {45, 50, 60} {45, 50, 70} {45, 50, 80} {45, 50, 100} {45, 50, 120} {45, 60, 70} {45, 60, 80} {45, 60, 100} {45, 60, 120} {45, 70, 80} {45, 70, 100} {45, 70, 120} {45, 80, 100} {45, 80, 120} {45, 100, 120} {50, 60, 70} {50, 60, 80} {50, 60, 100} {50, 60, 120} {50, 70, 80} {50, 70, 100} {50, 70, 120} {50, 80, 100} {50, 80, 120} {50, 100, 120} {60, 70, 80} {60, 70, 100} {60, 70, 120} {60, 80, 100} {60, 80, 120} {60, 100, 120} {70, 80, 100} {70, 80, 120} {70, 100, 120} or {80, 100, 120}.

In some embodiments, a set that defines a composition consisting of four Mesh sizes may include: {40, 45, 50, 60} {40, 45, 50, 70} {40, 45, 50, 80} {40, 45, 50, 100} {40, 45, 50, 120} {40, 45, 60, 70} {40, 45, 60, 80} {40, 45, 60, 100} {40, 45, 60, 120} {40, 45, 70, 80} {40, 45, 70, 100} {40, 45, 70, 120} {40, 45, 80, 100} {40, 45, 80, 120} {40, 45, 100, 120} {40, 50, 60, 70} {40, 50, 60, 80} {40, 50, 60, 100} {40, 50, 60, 120} {40, 50, 70, 80} {40, 50, 70, 100} {40, 50, 70, 120} {40, 50, 80, 100} {40, 50, 80, 120} {40, 50, 100, 120} {40, 60, 70, 80} {40, 60, 70, 100} {40, 60, 70, 120} {40, 60, 80, 100} {40, 60, 80, 120} {40, 60, 100, 120} {40, 70, 80, 100} {40, 70, 80, 120} {40, 70, 100, 120} {40, 80, 100, 120} {45, 50, 60, 70} {45, 50, 60, 80} {45, 50, 60, 100} {45, 50, 60, 120} {45, 50, 70, 80} {45, 50, 70, 100} {45, 50, 70, 120} {45, 50, 80, 100} {45, 50, 80, 120} {45, 50, 100, 120} {45, 60, 70, 80} {45, 60, 70, 100} {45, 60, 70, 120} {45, 60, 80, 100} {45, 60, 80, 120} {45, 60, 100, 120} {45, 70, 80, 100} {45, 70, 80, 120} {45, 70, 100, 120} {45, 80, 100, 120} {50, 60, 70, 80} {50, 60, 70, 100} {50, 60, 70, 120} {50, 60, 80, 100} {50, 60, 80, 120} {50, 60, 100, 120} {50, 70, 80, 100} {50, 70, 80, 120} {50, 70, 100, 120} {50, 80, 100, 120} {60, 70, 80, 100} {60, 70, 80, 120} {60, 70, 100, 120} {60, 80, 100, 120} or {70, 80, 100, 120}.

In some embodiments, a set that defines a composition consisting of five Mesh sizes may include: {40, 45, 50, 60, 70} {40, 45, 50, 60, 80} {40, 45, 50, 60, 100} {40, 45, 50, 60, 120} {40, 45, 50, 70, 80} {40, 45, 50, 70, 100} {40, 45, 50, 70, 120} {40, 45, 50, 80, 100} {40, 45, 50, 80, 120} {40, 45, 50, 100, 120} {40, 45, 60, 70, 80} {40, 45, 60, 70, 100} {40, 45, 60, 70, 120} {40, 45, 60, 80, 100} {40, 45, 60, 80, 120} {40, 45, 60, 100, 120} {40, 45, 70, 80, 100} {40, 45, 70, 80, 120} {40, 45, 70, 100, 120} {40, 45, 80, 100, 120} {40, 50, 60, 70, 80} {40, 50, 60, 70, 100} {40, 50, 60, 70, 120} {40, 50, 60, 80, 100} {40, 50, 60, 80, 120} {40, 50, 60, 100, 120} {40, 50, 70, 80, 100} {40, 50, 70, 80, 120} {40, 50, 70, 100, 120} {40, 50, 80, 100, 120} {40, 60, 70, 80, 100} {40, 60, 70, 80, 120} {40, 60, 70, 100, 120} {40, 60, 80, 100, 120} {40, 70, 80, 100, 120} {45, 50, 60, 70, 80} {45, 50, 60, 70, 100} {45, 50, 60, 70, 120} {45, 50, 60, 80, 100} {45, 50, 60, 80, 120} {45, 50, 60, 100, 120} {45, 50, 70, 80, 100} {45, 50, 70, 80, 120} {45, 50, 70, 100, 120} {45, 50, 80, 100, 120} {45, 60, 70, 80, 100} {45, 60, 70, 80, 120} {45, 60, 70, 100, 120} {45, 60, 80, 100, 120} {45, 70, 80, 100, 120} {50, 60, 70, 80, 100} {50, 60, 70, 80, 120} {50, 60, 70, 100, 120} {50, 60, 80, 100, 120} {50, 70, 80, 100, 120} or {60, 70, 80, 100, 120}.

In some embodiments, a set that defines a composition consisting of six Mesh sizes may include: {40, 45, 50, 60, 70, 80} {40, 45, 50, 60, 70, 100} {40, 45, 50, 60, 70, 120} {40, 45, 50, 60, 80, 100} {40, 45, 50, 60, 80, 120} {40, 45, 50, 60, 100, 120} {40, 45, 50, 70, 80, 100} {40, 45, 50, 70, 80, 120} {40, 45, 50, 70, 100, 120} {40, 45, 50, 80, 100, 120} {40, 45, 60, 70, 80, 100} {40, 45, 60, 70, 80, 120} {40, 45, 60, 70, 100, 120} {40, 45, 60, 80, 100, 120} {40, 45, 70, 80, 100, 120} {40, 50, 60, 70, 80, 100} {40, 50, 60, 70, 80, 120} {40, 50, 60, 70, 100, 120} {40, 50, 60, 80, 100, 120} {40, 50, 70, 80, 100, 120} {40, 60, 70, 80, 100, 120} {45, 50, 60, 70, 80, 100} {45, 50, 60, 70, 80, 120} {45, 50, 60, 70, 100, 120} {45, 50, 60, 80, 100, 120} {45, 50, 70, 80, 100, 120} {45, 60, 70, 80, 100, 120} or {50, 60, 70, 80, 100, 120}.

In some embodiments, a set that defines a composition consisting of seven Mesh sizes may include: {40, 45, 50, 60, 70, 80, 100} {40, 45, 50, 60, 70, 80, 120} {40, 45, 50, 60, 70, 100, 120} {40, 45, 50, 60, 80, 100, 120} {40, 45, 50, 70, 80, 100, 120} {40, 45, 60, 70, 80, 100, 120} {40, 50, 60, 70, 80, 100, 120} or {45, 50, 60, 70, 80, 100, 120}.

The above-referenced examples of Mesh sizes used to define a composition in accordance with the present invention are merely examples of some preferred embodiments, and one or more suitable Mesh size may be used to define a composition that is within the scope and spirit of the present invention.

Additionally, various suitable percentages of total volume pass-through may be associated with a given Mesh size of a set of Mesh sizes that defines a composition. Such percentages may include progressively smaller percents as the Mesh number increases (smaller Mesh number indicates larger sized holes in a mesh, whereas larger Mesh number indicates smaller sized holes in a mesh), but in some embodiments, the same percentage may be associated with subsequent progressive Mesh numbers.

In some embodiments, a smallest Mesh number of a set (largest sized holes in a mesh) may include ranges such as 95-100%, 90-100%, 85-100%, 80-100%, 75-100%, 70-100%, 90-95%, 85-95%, 80-95%, 75-95%, 70-95%, or the like.

In some embodiments, a second-smallest Mesh number of a set (second-largest sized holes in a mesh) may include ranges such as 60-85%, 50-85%, 40-85%, 60-80%, 50-80%, 40-80%, 60-75%, 50-75%, 40-75%, or the like.

In some embodiments, a third-smallest Mesh number of a set (third-largest sized holes in a mesh) may include ranges such as 40-65%, 30-65%, 20-65%, 40-60%, 30-60%, 20-60%, 40-55%, 30-55%, 20-55%, or the like.

In some embodiments, a fourth-smallest Mesh number of a set (fourth-largest sized holes in a mesh) may include ranges such as 1-10%, 1-15%, 1-20%, 5-10%, 5-15%, 5-20%, or the like.

In some embodiments, a fifth-smallest Mesh number of a set (fifth-largest sized holes in a mesh) may include ranges such as 0.1-1.0%, 0.1-2.0%, 0.1-3.0%, 0.1-4.0%, 0.1-5.0%, 0.5-1.0%, 0.5-2.0%, 0.5-3.0%, 0.5-4.0%, 0.5-5.0%, or the like.

The above referenced percentage examples are not intended to be limiting, and are simply examples of percentages in accordance with some preferred embodiments. Percentages may be any suitable amount in accordance with various embodiments, which satisfy the spirit and scope of the present invention. As discussed above one or more Mesh size may be used to define a composition and therefore a respective one or more percentage will be associated with the one or more Mesh size.

In various embodiments, the compositions discussed herein may be used to provide for time-release of desirable compounds in a human or animal subject. For example, in various embodiments, disclosed compositions may be used for time-release of ellagitannins that are present in a plant material that defines the composition. Ellagitannins are antioxidants that are part of a diverse class of hydrolyzable tannins and are a type of polyphenol formed primarily from the oxidative linkage of galloyl groups in 1,2,3,4,6-pentagalloyl glucose. Examples of ellagitannins are castalagin, castalin, casuarictin, grandinin, punicalagin, punicalin, roburin A, tellimagrandin II, terflavin B, vescalagin, and the like.

Ellagitannins are present in plant material such as seeds, and unexpectedly, by generating compositions with different sizes of portions of seeds, a time-release and controlled delivery system for ellagitannins can be produced. Moreover, in various embodiments, and unexpectedly, such a time-release and controlled delivery system for ellagitannins can be produced with minimal processing of the seeds.

For example, unlike conventional time-release and controlled delivery systems, where a desired compound is physically encased within a plastic from which it slowly escapes over a period of time by the processes of diffusion, in various embodiments a seed based time-release and controlled delivery system for ellagitannins can be produced without encasing the seeds in a substance or otherwise directly associating seed particles with a substance that creates time-release properties.

In another example, many other conventional time-release and controlled delivery systems, includes compositions where a desired compound is chemically reacted with a plastic to become a new substance and thus to become temporarily bio-unavailable. However, over time, the chemical linkage can be cleaved by the chemistry in the body of a subject to release the desired compound in its active form. In contrast, in various embodiments, a seed based time-release and controlled delivery system for ellagitannins can be produced without reacting seed particles with one or more compounds to limit, slow, or otherwise modify the bioavailability of the ellagitannins present in the seed particles.

Accordingly, various embodiments include compositions of variably sized seed particles that have been minimally processed to generate a time-release and controlled delivery system for ellagitannins that relies on the natural bioavailability of the ellagitannins in the seed particles. For example, various embodiments specifically exclude or are without processing steps described in the prior art. By selecting the sizes and amounts of seed particles in a composition as discussed above in a time-release and controlled delivery system for ellagitannins may be generated that provides time-release and controlled delivery of ellagitannins over a desired period of time, and with a desired release profile.

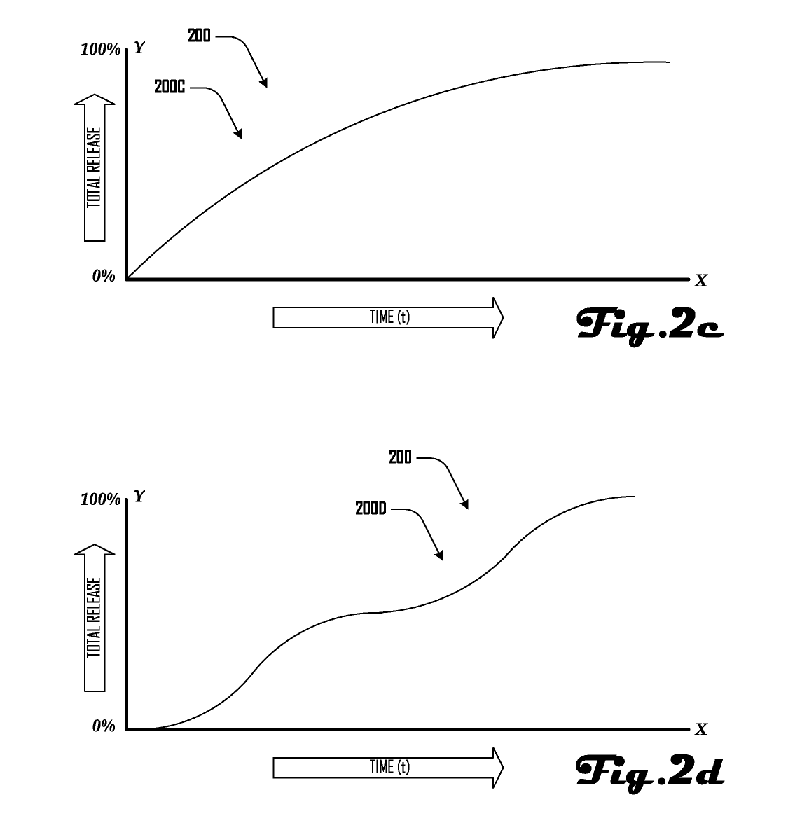

For example, FIGS. 2a-2d depict time-release profiles in accordance with some embodiments. These profiles are only intended to be illustrations of possible time-release profiles, and by selecting the sizes and amounts of seed particles 100 in a composition as discussed above, a desired time-release profile may be generated. Profiles may be linear, exponential, geometric, or irregular as desire. Time release may occur over various desired intervals including 1 hour, 2 hours, 3 hours, 6 hours, 12 hours, 24 hours, 36 hours, 48 hours, or the like.

For example, in some embodiments, larger seed particles may slowly release ellagitannins over a period of time because only ellagitannins on the outer surface area of a seed particle may be bio-available as these ellagitannins are processed by the digestive processes of a subject. Accordingly, in various embodiments, larger sized particles may be used to provide longer periods of ellagitannin time-release.

Generating and testing a composition with a desired time release profile may be done by generating a composition as discussed herein and measuring the release of ellagitannins over time in vitro under conditions that that simulate the digestive track of a subject. Accordingly, compositions that release a total desired amount of ellagitannins over a desired period of time, with a desired time-release profile may be generated and refined.

In some preferred embodiments, desired time-release compounds are ellagitannins As discussed herein, various plant materials may be used as a source for ellagitannins when generating ellagitannin time-release compositions as discussed herein. In some preferred embodiments, the seeds of cane-berries, including raspberries, blackberries, and the like may provide a source of variably sized seed particles for compositions disclosed herein. In some embodiments, Meeker raspberries are preferred. In various embodiments, the present compositions, systems and methods can apply to virgin seed or defatted seed (e.g., cold-pressed discharged material).

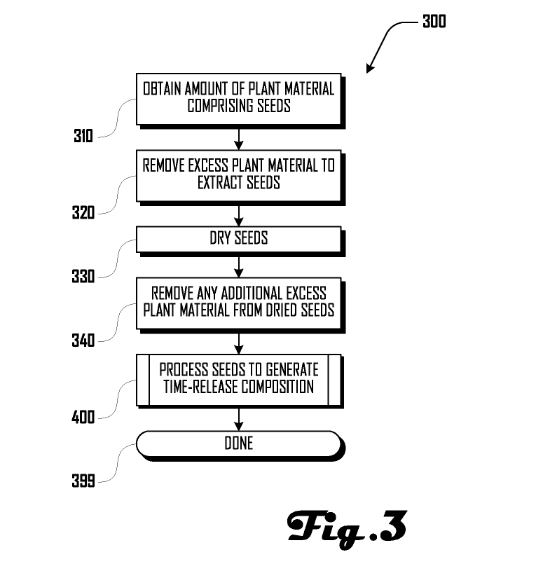

FIG. 3 is a block diagram of a method 300 for generating a time release composition from plant material comprising seeds, in accordance with an embodiment. The method begins in block 310 where an amount of plant material comprising seeds is obtained, and in block 320, excess plant material is removed to extract seeds. For example, such material may be picked cane-berries or waste material from making cane-berry juice. Such material may include stems, skin, and berry flesh in addition to seeds, and such non-seed material may selectively removed. In block 330 the seeds are dried, and in block 340, any additional undesired non-seed material is removed from the dried seeds. However, in some preferred embodiments, excess plant material is not removed.

For example, in some embodiments, it may be desirable to dry the berries until they have moisture content by weight of less than 20%, 15%, 10%, 5%, 1%, or the like. In a preferred embodiment, seeds are dried to a moisture content of less than 9% by weight. Seeds may be dried in any suitable conventional manner; however, in some preferred embodiments, seeds are dried in an oven at a temperature of about between 120° F. and 225° F. In one preferred embodiment, seeds are dried in an oven at a temperature of about 175° F. In some embodiments, it may be desirable to dry seeds in an atmosphere having at least about 80% nitrogen or other inert gas.

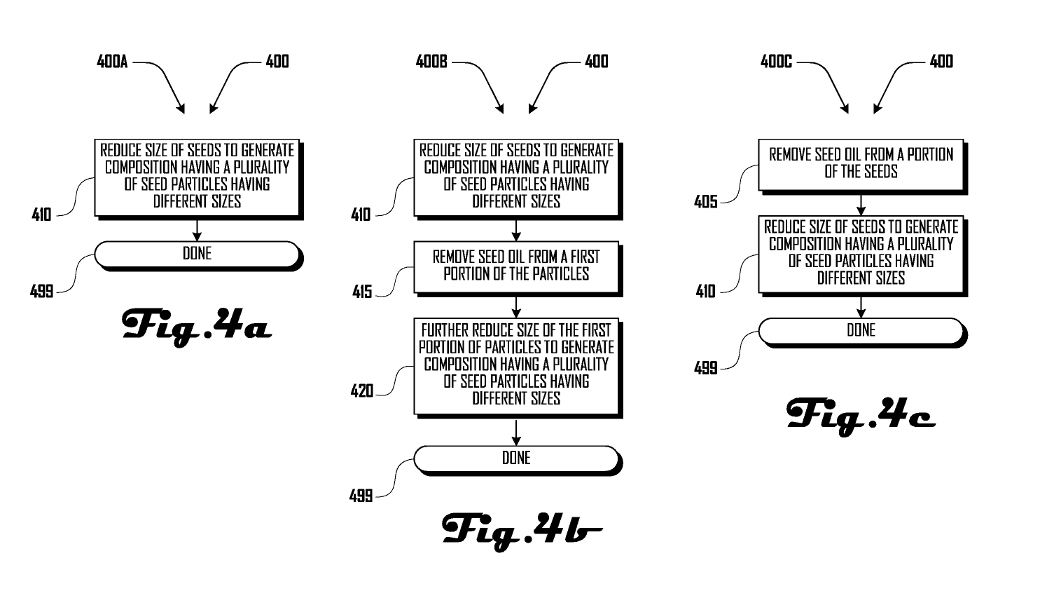

Returning to the method 300, in block 400, the seeds are processed to generate a time-release composition, and the method 300 is done in block 399. FIGS. 4a-4c are block diagrams of methods 400 for generating time release compositions from processed seeds, in accordance with some embodiments 400A, 400B, 400C.

Turning to FIG. 4a, this embodiment 400A of the method 400 begins in block 410 where the size of seeds is reduced to generate a composition having a plurality of seed particles having different sizes. The method is done in block 499. The size of seeds may be reduced in any suitable way including grinding, crushing, chopping or the like.

Seeds may comprise oils and it may be desirable to remove a portion of such oils from seeds in some embodiments. For example, it may be desirable to remove oils to reduce the weight of the seeds and thereby increase the ellagitannin content to weight ratio of the seeds. In some embodiments, removing oils from seeds may increase the bioavailability of ellagitannins in the seeds.

In further embodiments, it may be desirable to remove oils from seeds to facilitate making seed particles of smaller sizes. For example, when grinding seed particles to small sizes, oils may be released from the seeds which may result in undesirable clumping or binding of the seed particles to each other or to larger seed particles. Clumping may change the bioavailability of ellagitannins in the clumps because of reduced surface area exposure, and may undesirably effect the time-release profile of the composition. In some embodiments, where seed particles are finer than 80, 100, 120, 140, 170, 200, 230 or 270 Mesh, it may be desirable to remove oils from a portion of the seed particles.

Referring to FIG. 4b, oil is removed from only a portion of the seeds in one embodiment 400B. In block 410, the size of seeds is reduced to generate a composition having a plurality of seed particles having different sizes, and in block 415, seed oil is removed from a first portion of the particles. In block 420 the size of the first portion is further reduced to generate a composition having a plurality of seed particles having different sizes. The method 400B is done in block 499.

In contrast, and referring to FIG. 4c, seed oil is removed from all of the seeds in another embodiment 400C. In block 405, seed oil is removed from a portion of the seeds and in block 410, the size of seeds is reduced to generate a composition having a plurality of seed particles having different sizes. The method 400C is done in block 499.

Although FIGS. 4a-c depict seed oil being removed at certain times in a manufacturing process and removed from certain portion of the seeds, oil may be removed from any portion of seeds being processed in any suitable amount, at any suitable stage in a manufacturing or processing method. The present examples are not intended to be limiting. For example, in some embodiments, oil may be removed from seeds before the seeds are dried.

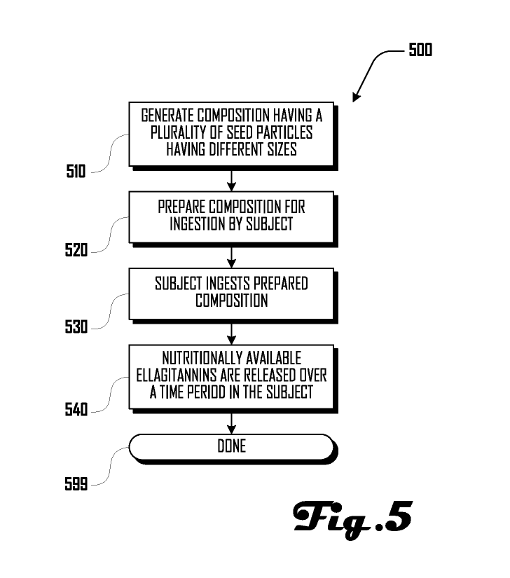

FIG. 5 is a block diagram of a method 500 for time release of ellagitannins in a subject. The method 500 begins in block 510, where a composition is generated having a plurality of seed particles 100 of different sizes as discussed herein. In block 520, the composition is prepared for ingestion by a subject and the subject ingests the prepared composition, in block 530. In some embodiments, preparation of a composition may include measuring a dosage of a prepared composition. In some embodiments, preparation may comprise packaging the composition in a pill or capsule form.

In block 540, nutritionally available ellagitannins are released over a time period in the subject, and in block 599, the method 500 is done. In various embodiments the ellagitannins may be nutritionally available in an amount of at least about 80%, 85%, 90%, 95%, 98% or 99% by weight. In a preferred embodiment, ellagitannins are nutritionally available at least about 95% by weight. In other words, of the total amount of ellagitannins present in an ingested composition, 95% will be released into the body of the subject over the release time period.

In addition to being used for time-release after ingestion by a subject, compositions discussed herein may also be used in cosmetics and external products. In some embodiments, the compositions may be used for time release of ellagitannins or other compounds on external body parts. In some embodiments, compositions disclosed herein may serve a function as an exfoliant in a cosmetic product. For example, in various embodiments, cosmetics having only natural seed-based exfoliants may be desirable because synthetic exfoliants such as plastic beads may be prohibited in some jurisdictions. Compositions comprising coarse seed particles may be desirable, including portions of a seed particle composition that do not pass through 35, 40, 45, 50, or 60 Mesh.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives.

What is claimed is:

1. A composition for time release of ellagitannins comprising:
a plurality of substantially dried seed particles having ellagitannins, the plurality of seed particles being of different sizes, including:
a first portion having a size operable to pass through a first Mesh size;
a second portion having a size operable to pass through a second Mesh size that is greater than the first Mesh size, the second portion being smaller than the first portion, and
a third portion having a size operable to pass through a third Mesh size that is greater than the second Mesh size, the third portion being smaller than the second portion.

2. The composition of claim 1, further comprising a fourth portion having a size operable to pass through a fourth Mesh size that is greater than the third Mesh size, the fourth portion being smaller than the third portion.

3. The composition of claim 2, further comprising a fifth portion having a size operable to pass through a fifth Mesh size that is greater than the fourth Mesh size, the fifth portion being smaller than the fourth portion.

4. The composition of claim 3, wherein the first, second, third, fourth and fifth Mesh sizes are at least about 20 units apart.

5. The composition of claim 3, wherein the first portion is about 95%-100% per volume; the second portion is about 60%-80% per volume; the third portion is about 40%-60% per volume; the fourth portion is about 1%-10% per volume; and the fifth portion is about 0.1% -1% per volume.

6. The composition of claim 3, wherein the first, second, third, fourth and fifth Mesh sizes are about 40, 60, 70, 100 and 200 respectively.

7. The composition of claim 1, wherein about 95% of the ellagitannin is nutritionally available to a human subject consuming the composition.

8. The composition of claim 1, wherein the composition is configured to provide time release of nutritionally available ellagitannin to a human subject consuming the composition.

9. The composition of claim 8, wherein the time release occurs over a period of at least 24 hours.

10. The composition of claim 1, wherein the composition consists essentially of the plurality of substantially dried seed particles.

11. The composition of claim 1, wherein the moisture content of the plurality of substantially dried seed particles is about 1%-9%.

12. The composition of claim 1, wherein at least one portion of the dried seed particles comprises seed particles that have been processed to reduce the seed oil content of the seed particles.

13. The composition of claim 1, wherein the seed particles substantially consist of berry seeds.

14. A cosmetic product comprising the composition of claim 1.

15. A nutraceutical product comprising the composition of claim 1.

16. A method of making a composition for time release of ellagitannins, the method comprising:
obtaining an amount of plant material having a substantial amount of ellagitannins;
reducing the size of at least a portion of the plant material to generate a composition having a plurality of particles being of different sizes, including:
a first portion having a size operable to pass through a first Mesh size;
a second portion having a size operable to pass through a second Mesh size that is greater than the first Mesh size, the second portion being smaller than the first portion, and
a third portion having a size operable to pass through a third Mesh size that is greater than the second Mesh size, the third portion being smaller than the second portion.

17. The method of claim 16, further comprising reducing the oil content of at least a portion of the plant material.

18. The composition of claim 3,
wherein the first, second, third Mesh sizes are at least about 20 units apart; and
wherein the first portion is about 95%-100% per volume; the second portion is about 60%-80% per volume; and the third portion is about 40%-60% per volume.

19. A method of time release of ellagitannins in a human subject, the method comprising:
obtaining suitable amount of a composition having a plurality of seed particles being of different sizes and including a substantial amount of ellagitannins, including:
a first portion having a size operable to pass through a first Mesh size;
a second portion having a size operable to pass through a second Mesh size that is greater than the first Mesh size, the second portion being smaller than the first portion, and
a third portion having a size operable to pass through a third Mesh size that is greater than the second Mesh size, the third portion being smaller than the second portion;
ingesting of the composition by a human subject, where the seed particles are without a coating and have not been treated to modify the nutritional availability of the ellagitannins; and
releasing, by the composition over a time period of at least 12 hours, nutritionally available amounts of ellagitannins to the body of the human subject.
